(19) United States
(12) Patent Application Publication
(10) Pub. No.: US 2025/0073089 A1
(43) Pub. Date: Mar. 6, 2025

(54) Diaper Device with Methane Sensor

(71) Applicant: David Whatley, Muskogee, OK (US)

(72) Inventor: David Whatley, Muskogee, OK (US)

(21) Appl. No.: 18/430,791

(22) Filed: Feb. 2, 2024

Related U.S. Application Data
(60) Provisional application No. 63/579,048, filed on Aug. 28, 2023.

(51) Int. Cl. A61F 13/15; A61F 13/42

(52) U.S. Cl. CPC A61F 13/15585; A61F 13/42; A61F 2013/424

(57) ABSTRACT

A diaper device with methane sensor is provided. The device is comprised of a diaper with an integrated methane gas sensor. The sensor has a threshold level for methane gas that is preferably reached when an infant defecates in the diaper. Once the threshold is met or exceeded, the sensor communicates with a mobile application via a transmitter to produce a notification on the smart device the application is installed on. As a result, a user is alerted that the infant's diaper needs changed via their smart device.

### CROSS-REFERENCE TO RELATED APPLICATION

The present application claims priority to, and the benefit of, U.S. Provisional Application No. 63/579,048, which was filed on Aug. 28, 2023, and is incorporated herein by reference in its entirety.

### FIELD OF THE INVENTION

The present invention relates generally to the field of diapers. More specifically, the present invention relates to a diaper device with methane sensor comprised of an infant, child, or adult diaper with an internal methane sensor that alerts a user via a mobile application when the diaper needs changed. Accordingly, the present disclosure makes specific reference thereto. Nonetheless, it is to be appreciated that aspects of the present invention are also equally applicable to other like applications, devices, and methods of manufacture.

### BACKGROUND

When an infant's need for a diaper change is overlooked, the ensuing consequences can go beyond just the immediate discomfort. Diaper rash, which may develop due to prolonged exposure to moisture and irritants, can be a painful experience for the child. This reddening and inflammation of the skin is not just a temporary ailment; it can become a recurrent issue if not addressed promptly.

Moreover, for those who might not have frequent interactions with infants, understanding and interpreting their subtle signals can be quite challenging. Babies, especially in their early months, communicate primarily through non-verbal cues. A slight change in their mood, restlessness, or even specific cries can indicate discomfort due to a soiled diaper. However, without keen observation and understanding, these signals can easily be missed, leading to prolonged periods in an uncomfortable state for the child.

Beyond the physical discomfort, neglecting timely diaper changes can also have emotional and psychological implications for the baby. Infants rely entirely on their caregivers for their basic needs. Consistently missing their signals for a clean diaper can lead to feelings of insecurity and mistrust. While they may not consciously remember these experiences as they grow older, such repeated instances can shape their emotional responses and attachment patterns as they age.

Therefore, there exists a long-felt need in the art for a device that helps a user know when an infant needs a diaper change. There also exists a long-felt need in the art for a diaper device with methane sensor. More specifically, there exists a long-felt need in the art for a diaper device with methane sensor wherein the device alerts a user when an infant needs a diaper change by detecting methane levels within a diaper. Further, there exists a long-felt need in the art for a diaper device with methane sensor wherein the device alerts a user when an infant needs a diaper change by detecting methane levels within a diaper wherein the device alerts a user even if the user is not immediately near the infant.

The subject matter disclosed and claimed herein, in one embodiment thereof, comprises a diaper device with methane sensor. The device is comprised of a diaper with an integrated methane gas sensor. The sensor has a threshold level for methane gas that is preferably reached when an infant defecates in the diaper. Once the threshold is met or exceeded, the sensor communicates with a mobile application via a transmitter to produce a notification on the smart device the application is installed on. In this manner, a user is alerted that the infant's diaper needs changed.

In this manner, the diaper device with methane sensor of the present invention accomplishes all the foregoing objectives and provides a device that helps a user in knowing when an infant needs a diaper change. More specifically, the device alerts a user when an infant needs a diaper change by detecting methane levels within a diaper. Further, the device does so even if the user is not immediately near the infant due to the mobile application.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a diaper device with methane sensor. The device is comprised of a diaper comprised of at least one sensor. The sensor is preferably a methane sensor that detects the presence of methane gas—an indication that the wearer of the diaper has defecated. Once a sufficient amount of methane is detected, a user is alerted via a mobile application.

The diaper may be any style, shape, size, and configuration of infant, child, or adult diaper known in the art. The diaper is preferably comprised of at least one pad. The pad is preferably made from an absorbent material. The diaper is also comprised of at least one sensor. The sensor is preferably a methane sensor that detects methane gas. The sensor is preferably enclosed in a casing that is waterproof to protect the sensor from damage from urine and feces. Further, the casing is preferably gas-permeable to allow the sensor to detect methane gas.

The sensor is comprised of a threshold level. The threshold level may be any specific level of any specific unit that measures methane gas (or any other gas). The threshold level is preferably configured so as to not cause the sensor to alert due to flatulence, and only alert to defecation within the diaper. Once the threshold level has been detected and/or exceeded, the sensor communicates with a mobile application. The application then notifies a user when the sensor has detected a methane level within the diaper at or above the threshold level. In this manner, the user is aware the infant's diaper needs changed.

The present invention is also comprised of a method of using the device. First, a device is provided comprised of a diaper comprised of at least one methane sensor and a mobile application. Then, a user places the diaper on an infant, child, or adult. Next, a user downloads a mobile application to a smart device. Once the mobile application alerts a user that the infant, child, or adult needs a diaper change via an alert, the user can then change the diaper as needed.

Accordingly, the diaper device with methane sensor of the present invention is particularly advantageous as it provides a device that helps a user know when an infant needs a diaper change. More specifically, the device alerts a user when an infant needs a diaper change by detecting methane levels within a diaper. Further, the device does so even if the user is not immediately near the infant due to the mobile application. In this manner, the diaper device with methane sensor provides a solution to easily know when an infant needs a diaper change.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a diaper device with methane sensor of the present invention in accordance with the disclosed architecture;

FIG. 2 illustrates a graphical view of a mobile application of one potential embodiment of a diaper device with methane sensor of the present invention in accordance with the disclosed architecture; and

FIG. 3 illustrates a flowchart of a method of using one potential embodiment of a diaper device with methane sensor of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that helps a user know when an infant needs a diaper change. There also exists a long-felt need in the art for a diaper device with methane sensor. More specifically, there exists a long-felt need in the art for a diaper device with methane sensor wherein the device alerts a user when an infant needs a diaper change by detecting methane levels within a diaper. Further, there exists a long-felt need in the art for a diaper device with methane sensor wherein the device alerts a user when an infant needs a diaper change by detecting methane levels within a diaper wherein the device alerts a user even if the user is not immediately near the infant.

The present invention, in one exemplary embodiment, is comprised of a diaper device with methane sensor. The device is comprised of a diaper comprised of at least one sensor. The sensor is preferably a methane sensor that detects the presence of methane gas—an indication that the wearer of the diaper has defecated. Once a sufficient amount of methane is detected, a user is alerted via a mobile application.

The diaper may be any style, shape, size, and configuration of infant, child, or adult diaper known in the art and is preferably comprised of at least one pad. The pad is preferably made from an absorbent material. The diaper is also comprised of at least one sensor which is preferably a methane sensor that detects methane gas. The sensor is preferably enclosed in a casing that is waterproof to protect the sensor from damage from urine and feces. Further, the casing is preferably gas-permeable to allow the sensor to detect methane gas.

The sensor is comprised of a threshold level. The threshold level may be any specific level of any specific unit that measures methane gas (or any other gas). The threshold level is preferably configured to not cause the sensor to alert due to flatulence, and only alert to defecation within the diaper. Once the threshold level has been detected and/or exceeded, the sensor communicates with a mobile application. The application then notifies a user when the sensor has detected a methane level within the diaper at or above the threshold level. In this manner, the user is aware the infant's diaper needs changed.

The present invention is also comprised of a method of using the device. First, a device is provided comprised of a diaper comprised of at least one methane sensor and a mobile application. Then, a user places the diaper on an infant, child, or adult. Next, a user downloads a mobile application to a smart device. Once the mobile application alerts a user that the infant, child, or adult needs a diaper change via an alert, the user can then change the diaper as needed.

Accordingly, the diaper device with methane sensor of the present invention is particularly advantageous as it provides a device that helps a user know when an infant needs a diaper change. More specifically, the device alerts a user when an infant needs a diaper change by detecting methane levels within a diaper. Further, the device does so even if the user is not immediately near the infant due to the mobile application. In this manner, the diaper device with methane sensor provides a solution to easily know when an infant needs a diaper change.

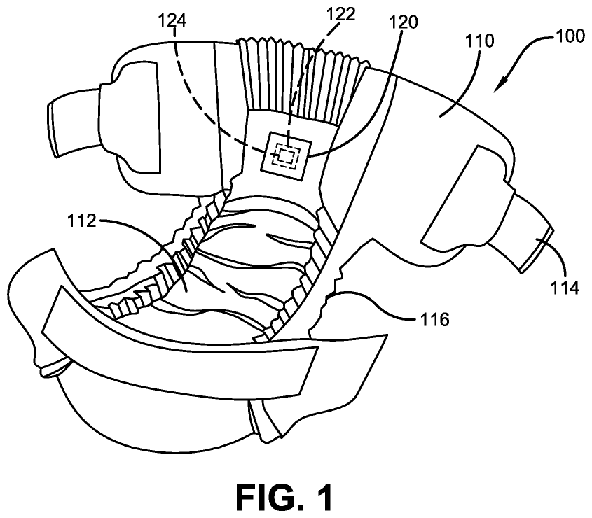

Referring initially to the drawings, FIG. 1 illustrates a perspective view of one potential embodiment of a diaper device with methane sensor 100 of the present invention in accordance with the disclosed architecture. The device 100 is comprised of a diaper 110 and at least one sensor 120. The sensor 120 is preferably a methane sensor that detects the presence of methane gas—an indication that the wearer of the diaper 110 has defecated. Once a sufficient amount of methane is detected, a user is alerted via a mobile application 130.

The device 100 is comprised of a diaper 110. The diaper 110 may be any style, shape, size, and configuration of infant, child, or adult diaper known in the art. The diaper 110 is preferably comprised of at least one pad 112. The pad 112 is preferably made from an absorbent material such as, but not limited to, polyester, cotton, bamboo, hemp, wood pulp, etc. In one embodiment, the pad 112 may be scented with any scent.

The diaper 110 preferably has at least one fastener 114. The fastener 114 allows the diaper 110 to be secured around the body of the user. The fastener 114 is preferably, but not limited to, an adhesive fastener. The diaper 110 may also have at least one leg opening 116.

The diaper 110 is comprised of at least one sensor 120. The sensor 120 is preferably positioned inside the diaper on, in, or near the pad 112. The sensor 120 is preferably a methane sensor that detects methane gas.

The sensor 120 is comprised of a threshold level. The threshold level may be any specific level of any specific unit that measures methane gas (or any other gas). The threshold level is preferably configured so as to not cause the sensor 120 to alert due to flatulence, and only alert to defecation within the diaper 110.

Once the threshold level has been detected and/or exceeded, at least one transmitter 124 of the sensor 120 communicates with a mobile application 130. The transmitter 124 is preferably a wireless communication sensor such as, but not limited to, Wi-Fi, Bluetooth, etc.

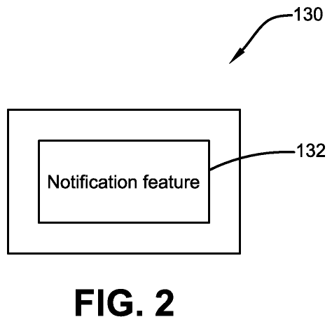

The mobile application 130 can be downloaded on any smart device such as, but not limited to, a computer, smartphone, tablet, etc. The application 130 is comprised of a notification feature 132, as seen in FIG. 2. The feature 132 notifies a user when sensor 120 has detected a methane level within the diaper 110 at or above the threshold level. The notification may be in the form of, but is not limited to an email, a text, a phone call, an audible alert, a vibration alert, a push notification, etc. In this manner, the user knows the infant's diaper 110 needs to be changed.

The sensor 120 is preferably enclosed in a casing 122. The casing 122 is preferably waterproof to protect the sensor 120 from damage from urine and feces. Further, the casing 122 is preferably gas permeable to allow the sensor 120 to detect methane gas.

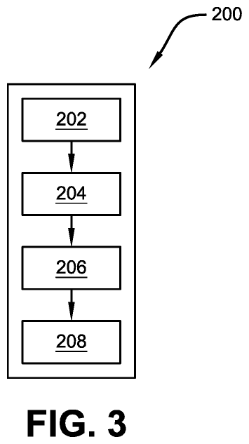

The present invention is also comprised of a method of using 200 the device 100, as seen in FIG. 3. First, a device 100 is provided comprised of a diaper 110 comprised of at least one methane sensor 120 and a mobile application 130 [Step 202]. Then, a user places the diaper 110 on an infant, child, or adult [Step 204]. Next, a user downloads a mobile application 130 to a smart device [Step 206]. Once the mobile application 130 alerts a user that the infant, child, or adult needs a diaper change via an alert, the user can then change the diaper 110 as needed [Step 208].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “diaper device with methane sensor” and “device” are interchangeable and refer to the diaper device with methane sensor 100 of the present invention.

Notwithstanding the foregoing, the diaper device with methane sensor 100 of the present invention and its various components can be of any suitable size and configuration as is known in the art without affecting the overall concept of the invention, provided that they accomplish the above-stated objectives. One of ordinary skill in the art will appreciate that the size, configuration, and material of the diaper device with methane sensor 100 as shown in the FIGS. are for illustrative purposes only, and that many other sizes and shapes of the diaper device with methane sensor 100 are well within the scope of the present disclosure. Although the dimensions of the diaper device with methane sensor 100 are important design parameters for user convenience, the diaper device with methane sensor 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A diaper device with methane sensor comprising:
a diaper comprised of a pad and a fastener;
a mobile application comprised of a notification feature; and
a sensor positioned within the diaper, the sensor comprised of a transmitter in wireless electrical communication with the mobile application.

2. The diaper device with methane sensor of claim 1, wherein the sensor is comprised of a methane sensor.

3. The diaper device with methane sensor of claim 2, wherein the methane sensor is comprised of a methane threshold level.

4. The diaper device with methane sensor of claim 3, wherein once the methane threshold level has been met or is exceeded, the notification feature sends a notification.

5. The diaper device with methane sensor of claim 1, wherein the notification is comprised of an email, a text, a phone call, an audible alert, a vibration alert, or a push notification.

6. A diaper device with methane sensor comprising:
a diaper comprised of a pad and a fastener;
a mobile application comprised of a notification feature; and
a methane sensor surrounded by a casing, wherein the methane sensor is positioned within the diaper and is comprised of a transmitter in wireless electrical communication with the mobile application.

7. The diaper device with methane sensor of claim 6, wherein the casing is comprised of a waterproof casing.

8. The diaper device with methane sensor of claim 6, wherein the casing is comprised of a gas-permeable casing.

9. The diaper device with methane sensor of claim 6, wherein the transmitter is comprised of a Wi-Fi transmitter.

10. The diaper device with methane sensor of claim 6, wherein the transmitter is comprised of a Bluetooth transmitter.

11. The diaper device with methane sensor of claim 6, wherein the methane sensor is comprised of a methane threshold level.

12. The diaper device with methane sensor of claim 11, wherein once the methane threshold level has been met or is exceeded, the notification feature sends a notification.

13. The diaper device with methane sensor of claim 12, wherein the notification is comprised of an email, a text, a phone call, an audible alert, a vibration alert, or a push notification.

14. The diaper device with methane sensor of claim 6, wherein the diaper is comprised of an infant diaper.

15. The diaper device with methane sensor of claim 6, wherein the diaper is comprised of a child diaper.

16. The diaper device with methane sensor of claim 6, wherein the diaper is comprised of an adult diaper.

17. A method of using a diaper device with methane sensor, the method comprising the following steps:
providing a diaper device with methane sensor comprised of a diaper comprised of a methane sensor and a mobile application;
placing the diaper on an infant, a child, or an adult; and
and changing the diaper once the mobile application has produced a notification.

18. The diaper device with methane sensor of claim 17, wherein the methane sensor is comprised of a methane threshold level.

19. The diaper device with methane sensor of claim 18, wherein once the methane threshold level has been met or is exceeded, the notification is sent.

20. The diaper device with methane sensor of claim 17, wherein the notification is comprised of an email, a text, a phone call, an audible alert, a vibration alert, or a push notification.
